(19) United States
(12) Patent Application Publication
(10) Pub. No.: US 2022/0111578 A1
(43) Pub. Date: Apr. 14, 2022

(54) KINK-RESISTANT ELECTROSPUN FIBER ASSEMBLIES AND METHODS OF MAKING THE SAME

(71) Applicant: Nanofiber Solutions, LLC, Dublin, OH (US)

(72) Inventors: Jed K. Johnson, London, OH (US); Kevin Nelson, Hilliard, OH (US); Tyler Groehl, Dublin, OH (US)

(73) Assignee: Nanofiber Solutions, LLC, Dublin, OH (US)

(21) Appl. No.: 17/500,192

(22) Filed: Oct. 13, 2021

Related U.S. Application Data
(60) Provisional application No. 63/090,962, filed on Oct. 13, 2020.

(51) Int. Cl. B29C 53/30

(52) U.S. Cl. CPC B29C 53/30; B29L 2031/7532

(57) ABSTRACT

Systems and methods of fabricating corrugated electrospun fiber assemblies are disclosed herein. The method can include placing an electrospun fiber scaffold on a corrugation rod, wherein the corrugation rod comprises a helical structure; applying a monofilament fiber about the electrospun fiber scaffold and the corrugation rod from a dispenser as the dispenser is translated longitudinally and the corrugation rod is rotated such that the monofilament fiber is wrapped about the electrospun fiber scaffold at a defined threads per inch (TPI) to form a wrapped electrospun fiber assembly; and longitudinally compressing the corrugated electrospun fiber assembly until it has been compressed from a first length to a second length to form the corrugated electrospun fiber assembly. The corrugated electrospun fiber assemblies can be kink-resistant as compared to conventional electrospun fiber scaffolds. The corrugated electrospun fiber assemblies can be used in, for example, biological applications within a subject.

### CROSS-REFERENCE TO RELATED APPLICATION

The present application claims priority to U.S. Provisional Patent Application No. 63/090,962, titled KINK-RESISTANT ELECTROSPUN FIBER ASSEMBLIES AND METHODS OF MAKING THE SAME, filed Oct. 13, 2020, which is hereby incorporated by reference herein in its entirety.

### BACKGROUND

Kink resistance is an important characteristic of any electrospun structure that may need to bend, coil, or flex for a given application. Kink resistance determines the degree to which an electrospun structure may be bent or formed before kinking. A kink within an electrospun structure may reduce, slow, occlude, or prevent the flow of a substance through the electrospun structure.

Kink resistance may be particularly important for electrospun structures intended for use within biological organisms. In a subject's body, for example, the kinking of a luminal organ may reduce or prevent the flow of vital substances such as blood, gasses, or waste products, which could lead to serious illness, injury, or death.

Electrospun structures comprising electrospun fibers, which may be used to replace such luminal organs, are often long, uniform cylindrical structures with low kink resistance. This low kink resistance may be attributed to a lack of regions that are able to expand and contract.

### SUMMARY

In an embodiment, a method of fabricating a corrugated electrospun fiber assembly, the method comprising: placing an electrospun fiber scaffold on a corrugation rod; applying a monofilament fiber about the electrospun fiber scaffold and the corrugation rod from a dispenser as the dispenser is translated longitudinally and the corrugation rod is rotated such that the monofilament fiber is wrapped about the electrospun fiber scaffold at a defined threads per inch (TPI) to form a wrapped electrospun fiber assembly; and longitudinally compressing the corrugated electrospun fiber assembly until it has been compressed from a first length to a second length to form the corrugated electrospun fiber assembly.

In some embodiments of the method, the dispenser is coupled to a linear actuator configured to translate the dispenser longitudinally; and the corrugation rod is coupled to a rotary actuator configured to rotate the corrugation rod.

In some embodiments of the method, applying the monofilament fiber about the electrospun fiber scaffold comprises controlling the linear actuator and the rotary actuator to wrap the monofilament fiber about the electrospun fiber scaffold at the defined TPI and a controlled tension.

In some embodiments of the method, the defined TPI comprises from about 2 TPI to about 20 TPI.

In some embodiments of the method, the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

In some embodiments of the method, the method further comprises electrospinning a polymer to form the electrospun fiber scaffold.

In some embodiments of the method, the polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

In some embodiments of the method, the second length is from about 10% to about 90% of the first length.

In some embodiments of the method, the monofilament fiber has a diameter from about 20 μm to about 1000 μm.

In an embodiment, a corrugated electrospun fiber assembly comprising: an electrospun fiber scaffold; and a monofilament fiber wound about the electrospun fiber scaffold in a helical shape at a defined threads per inch (TPI) and a defined tension such that the electrospun fiber scaffold bulges between windings of the monofilament fiber after being compressed to form a corrugated surface.

In some embodiments of the corrugated electrospun fiber assembly, the defined TPI comprises from about 2 TPI to about 20 TPI.

In some embodiments of the corrugated electrospun fiber assembly, the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

In some embodiments of the corrugated electrospun fiber assembly, the electrospun fiber scaffold comprises a polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

In some embodiments of the corrugated electrospun fiber assembly, the monofilament fiber has a diameter from about 20 μm to about 1000 μm.

In an embodiment, a system for fabricating a corrugated electrospun fiber assembly, the system comprising: a corrugation rod comprising a helical structure, the corrugation rod configured to receive an electrospun fiber scaffold thereon; a rotary actuator configured to rotate the corrugation rod; a dispenser configured to dispense a monofilament fiber at a controlled tension; a linear actuator configured to linearly translate the dispenser; and a controller coupled to the rotary actuator and the linear actuator, the controller configured to control movement of at least one of the rotary actuator or the linear actuator to cause the monofilament fiber to wind about the electrospun fiber scaffold in a helical shape having a defined threads per inch (TPI).

In some embodiments of the system, the defined TPI comprises from about 2 TPI to about 20 TPI.

In some embodiments of the system, the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

In some embodiments of the system, the electrospun fiber scaffold comprises a polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

In some embodiments of the system, the monofilament fiber has a diameter from about 20 μm to about 1000 μm.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention.

FIG. 1 illustrates a system for fabricating a kink-resistant corrugated electrospun fiber assembly in accordance with one or more embodiments of the present disclosure.

FIG. 2 illustrates an electrospun fiber scaffold placed on a corrugation rod and wrapped with a monofilament fiber using the system illustrated in FIG. 1 in accordance with one or more embodiments of the present disclosure.

FIG. 3A illustrates a corrugated electrospun fiber assembly in an uncompressed state in accordance with one or more embodiments of the present disclosure.

FIG. 3B illustrates a corrugated electrospun fiber assembly in a compressed state in accordance with one or more embodiments of the present disclosure.

FIG. 4 illustrates a standard cylindrical electrospun structure with low kink resistance, as demonstrated by the bend and resulting occlusion shown therein.

FIG. 5 illustrates a corrugated electrospun fiber assembly that may be flexed considerably without kinking in accordance with one or more embodiments of the present disclosure.

FIG. 6 illustrates a graph demonstrating the compliance of standard cylindrical electrospun structures compared to the compliance of spirally configured electrospun structures with the same diameter and wall thickness in accordance with one or more embodiments of the present disclosure.

FIG. 7 illustrates a corrugated electrospun fiber assembly implanted in vivo in accordance with one or more embodiments of the present disclosure.

### DETAILED DESCRIPTION

The electrospun structures and associated methods disclosed herein may be used to form luminal electrospun fiber structures with thick and thin regions, or peaks and valleys, occurring periodically throughout the length of the electrospun structure. In some embodiments, the thin regions, or valleys, may have the ability to expand and contract, while the thick regions, or peaks, may maintain the electrospun structure's strength circumferentially. In some embodiments, the resulting electrospun structure may have a spiral configuration, thereby allowing it to withstand high degrees of bending, flexing, and coiling without kinking.

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50% means in the range of 40% to 60%.

As used herein, the term “subject” includes, but is not limited to, humans, non-human vertebrates, and animals such as wild, domestic, and farm animals. In some embodiments, the term “subject” refers to mammals. In some embodiments, the term “subject” refers to humans.

As used herein, the term “kink radius” means the minimum radius of the inside curvature of an object to which the object can be bent without kinking it. Generally speaking, a smaller kink radius corresponds to greater material flexibility because as the radius of curvature decreases, the amount of scaffold curvature increases.

### Electrospinning

Electrospinning is a method which may be used to process a polymer solution into a structure, such as a fiber. In embodiments wherein the diameter of the resulting fiber is on the nanometer scale, the fiber may be referred to as a nanofiber. Fibers may be formed into a variety of shapes by using a range of receiving surfaces, such as mandrels, molds, or collectors. In some embodiments, a flat shape, such as a sheet or sheet-like fiber structure, a fiber scaffold and/or tube, or a tubular lattice, may be formed by using a substantially round or cylindrical mandrel. The resulting fiber molds or shapes may be used in many applications, including the repair or replacement of biological structures. In some embodiments, the resulting structure (e.g., a fiber or fiber scaffold) may be implanted into a biological organism or a portion thereof.

Electrospinning methods may involve spinning a structure (e.g., a fiber) from a polymer solution by applying a high DC voltage potential between a polymer injection system and a receiving surface. In some embodiments, one or more charges may be applied to one or more components of an electrospinning system. In some embodiments, a charge may be applied to the receiving surface, the polymer injection system, the polymer solution, or combinations or portions thereof. Without wishing to be bound by theory, as the polymer solution is ejected from the polymer injection system, it is thought to be destabilized due to its exposure to a charge. The destabilized solution may then be attracted to a charged receiving surface. As the destabilized solution moves from the polymer injection system to the receiving surface, its solvents may evaporate and the polymer may stretch, leaving a long, thin fiber that is deposited onto the receiving surface. The polymer solution may form a Taylor cone as it is ejected from the polymer injection system and exposed to a charge. Further, polymers can be electrospun in a variety of different structures, including fibers, fibrous scaffolds, strips, patches, sheets, or shapes corresponding to anatomical structures. Still further, the structures can be electrospun using one or multiple polymers.

In some embodiments, multiple polymer types can be electrospun with each other to form structures in a process referred to as “co-electrospinning.” In co-electrospinning, two or more polymer solutions (containing the same or different polymer types) are ejected from different outlets and simultaneously electrospun with each other to form the resultant structure. Co-electrospinning creates two different fibers formed from the different polymer solutions that are intertwined with each other. The co-electrospun polymers can be spun from the same or different polymer solutions. The co-electrospun polymer types can have the same or different degradation rates. As one example, a first polymer type having a first degradation rate could be co-electrospun with a second polymer type having a second degradation rate, thereby creating an electrospun structure providing a time released profile that releases one or more pharmaceuticals contained within the different polymer types based on the differing degradation rates of the polymer types.

In some embodiments, multiple polymer types can be electrospun with each other to form structures in processes referred to as “coaxial electrospinning” or “multiaxial electrospinning.” In coaxial or multiaxial electrospinning, two or more polymer solutions (containing the same or different polymer types) are rejected from the same outlet and electrospun with each other to form the resultant structure. Coaxial or multiaxial electrospinning creates a single fiber composed of the different polymer types that has a core-shell structure. The coaxially or multi-axially electrospun polymer types can have the same or different degradation rates. As one example, a first polymer type having a first degradation rate could be coaxially or multi-axially electrospun with a second polymer type having a second degradation rate, thereby creating an electrospun structure providing a time released profile that releases one or more pharmaceuticals contained within the different polymer types based on the differing degradation rates of the polymer types.

In some embodiments, the co-electrospinning and coaxial or multiaxial electrospinning techniques described above could also be used in combination with each other. For example, coaxial polymers or fibers could be co-electrospun with each other. Accordingly, the various techniques described above can be used to electrospun structures having various timed release profiles for pharmaceuticals (which are described below) contained therein.

### Polymer Injection System

A polymer injection system may include any system configured to eject some amount of a polymer solution into an atmosphere to permit the flow of the polymer solution from the injection system to the receiving surface. In some embodiments, the polymer injection system may deliver a continuous or linear stream with a controlled volumetric flow rate of a polymer solution to be formed into a structure (e.g., a fiber). In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with one or more syringes containing one or more different polymer solutions. In some embodiments, the polymer injection system could include a rotating drum that dips into the polymer solution and ejects the solution as the drum rotates. In some embodiments, the polymer injection system could include a wire-based electrospinning system. In some embodiments, a charge may be applied to the polymer injection system, or to a portion thereof. In some embodiments, a charge may be applied to a needle or needle-like component of the polymer injection system. In one particular embodiment, the polymer injection system could include a wire electrode-based polymer injection system, such as the NS 8S1600U electrospinning production line available from ELMARCO®.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N-dimethylformamide, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution and/or the resultant electrospun polymer fiber(s) may also include additional materials. Non-limiting examples of such additional materials may include bacteriostatic agents, radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, or any other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, polypyrrole, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternative fibers may be composed of polymers that may be absorbed or bio-degraded over time. Such fibers may act as an initial template or scaffold during a healing process and/or for the repair or replacement of organs and/or tissues. These organ or tissue templates or scaffolds may degrade in vivo once the subject has exhibited a degree of healing and/or once the tissues or organs have been replaced or repaired by natural structures and cells. It may be further understood that a polymer solution and its resulting electrospun fiber(s) may be composed of more than one type of polymer, and that each polymer therein may have a specific characteristic, such as stability, or biodegradability.

### Applying Charges to Electrospinning Components

In an electrospinning system, one or more charges may be applied to one or more components, or portions of components, such as, for example, a receiving surface, a polymer injection system, a polymer solution, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to the polymer solution, or portions thereof. In some embodiments, a negative charge may be applied to the polymer solution, or portions thereof. In some embodiments, the polymer solution, or portions thereof, may be grounded. In some embodiments, a positive charge may be applied to the receiving surface, or portions thereof. In some embodiments, a negative charge may be applied to the receiving surface, or portions thereof. In some embodiments, the receiving surface, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −30 kV to about 100 kV, including endpoints. In some non-limiting examples, the charge applied to any component of the electrospinning system, or portions thereof, may be about −30 kV, about −25 kV, about −20 kV, about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, about 40 kV, about 50 kV, about 60 kV, about 70 kV, about 80 kV, about 90 kV, about 100 kV, or any range between any two of these values, including endpoints. In some embodiments, any component of the electrospinning system, or portions thereof, may be grounded.

### Receiving Surface Movement During Electrospinning

During electrospinning, in some embodiments, the receiving surface may move with respect to the polymer injection system. In some embodiments, the polymer injection system may move with respect to the receiving surface. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the receiving surface may be in contact with or mounted upon a support structure that may be moved using one or more motors or motion control systems. The pattern of the electrospun structure deposited on the receiving surface may depend upon the one or more motions of the receiving surface with respect to the polymer injection system. In some embodiments, the receiving surface may be configured to rotate about its long axis. In one non-limiting example, a receiving surface having a rotation rate about its long axis that is faster than a translation rate along a linear axis may result in a nearly helical deposition of an electrospun fiber, forming windings about the receiving surface. In another example, a receiving surface having a translation rate along a linear axis that is faster than a rotation rate about a rotational axis may result in a roughly linear deposition of an electrospun fiber along a linear extent of the receiving surface. In some embodiments, the electrospinning system could include a roller electrospinning system.

### System for Fabricating Corrugated Electrospun Fiber Assemblies

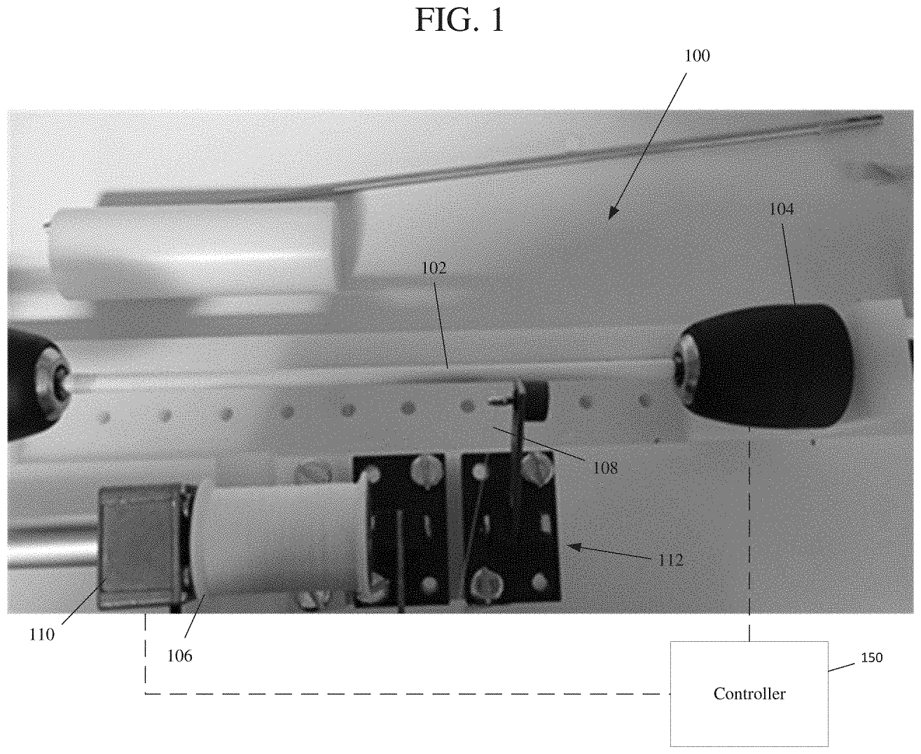

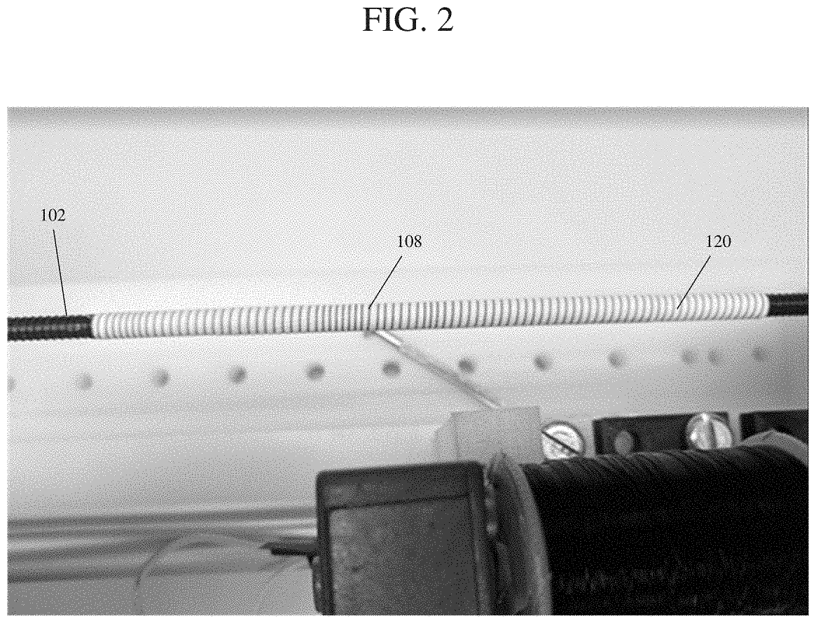

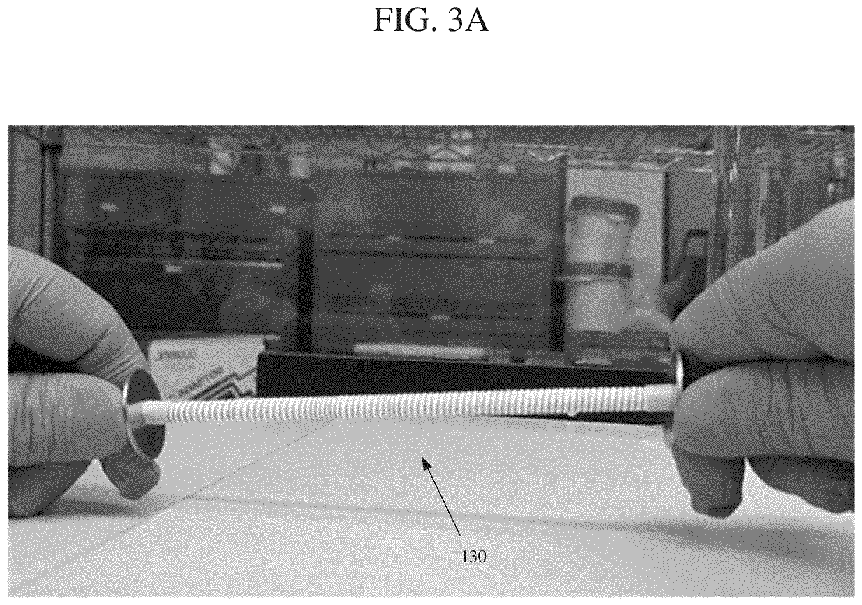

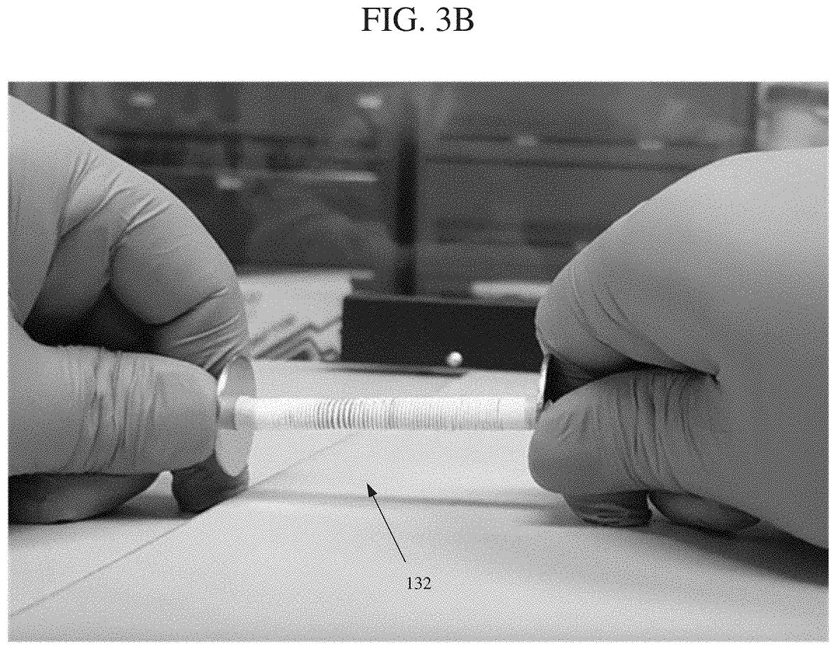

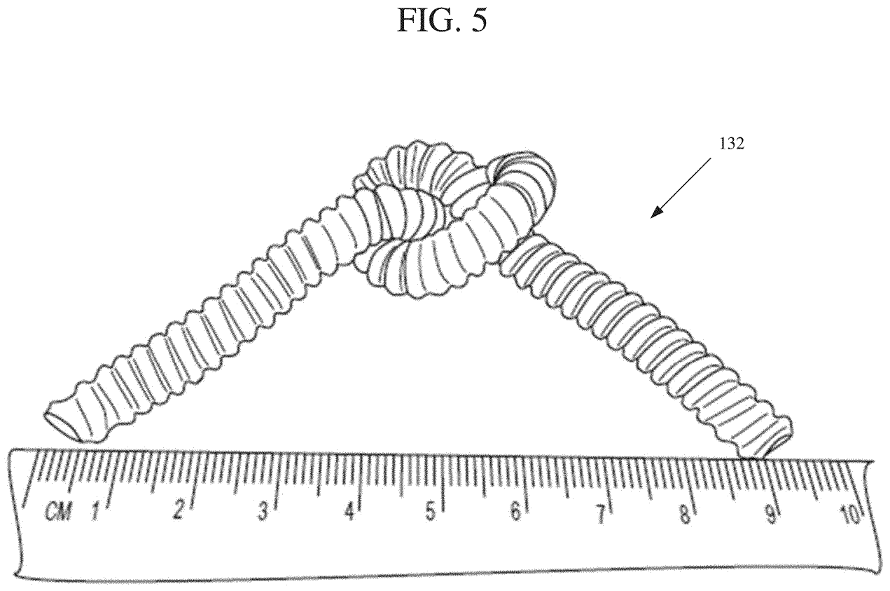

Referring now to FIGS. 1 and 2, in some embodiments, a corrugation system 100 may be configured to form a kink-resistant, corrugated electrospun fiber assembly. In some embodiments, the corrugation system 100 can include a corrugation rod 102 that is configured to support the electrospun fiber scaffold 120 and a monofilament fiber 108 during the corrugation process, which is described below. In one embodiment, the corrugation rod 102 can include a smooth surface. In another embodiment, the corrugation rod 102 can include a helical surface or helical structure thereabout, such as a helical ceramic structure, a helical plastic structure, and the like. In some embodiments, the corrugation rod 102 and the spiral structure may be concentrically configured. The corrugation rod 102 can be sized, shaped, or otherwise configured to receive an electrospun fiber scaffold 120 that has been fabricated using, for example, the techniques described above. FIG. 2, for example, illustrates the electrospun fiber scaffold 120 received on the corrugation rod 102. The corrugation rod 102 can be coupled to a first actuator 104 that is configured to translate and/or rotate the corrugation rod 102. In one embodiment, the first actuator 104 can include a rotary actuator that is configured to rotate the corrugation rod 102. In some embodiments, the electrospinning system 100 can further include a dispenser 106 that is configured to dispense a monofilament fiber 108 therefrom. The monofilament fiber 108 can be variety of different materials, including polydioxanone, Nylon, chromic (e.g., chromic gut sutures), glycolide, polypropylene, poliglecaprone, silk, gut (e.g., catgut), polydiaxanone, or polyglactin, for example. In one embodiment, the dispenser 106 can include a spool about which the monofilament fiber 108 is wound. The dispenser 106 can be coupled to a second actuator 110 that is configured to translate and/or rotate the dispenser 106. In one embodiment, the second actuator 110 can include a linear actuator that is configured to linearly translate the dispenser 106. In operation, the monofilament fiber 108 can be secured to or about the electrospun fiber scaffold 120, and the first actuator 104 and the second actuator 110 can be controlled to collectively translate and/or rotate the corrugation rod 102 and the dispenser 106 relative to each other to cause the monofilament fiber 108 to wrap or wind about the electrospun fiber scaffold 120. The monofilament fiber 108 wrapped about the electrospun fiber scaffold 120 thereby forms a corrugated surface on the electrospun fiber scaffold 120 (i.e., a surface having ridges or grooves). The electrospun fiber scaffold 120 having the monofilament fiber 108 wrapped therearound can be collectively referred to as a corrugated electrospun fiber assembly 132 (FIGS. 3B and 5).

In some embodiments, the corrugation system 100 can further include a tensioning assembly 112 that is configured to apply tension to the monofilament fiber 108 as it is applied about the electrospun fiber scaffold 120. In one embodiment, the tensioning assembly 112 can include one or more pulleys. In this embodiment, the monofilament fiber 108 can be drawn through the pulley assembly and attached to a location on the electrospun fiber scaffold 120 such that the pulley assembly provides a desired tension weight to the monofilament fiber 108 during fabrication of the corrugated electrospun fiber assembly 132.

In some embodiments, the monofilament fiber 108 can form a helical shape as it is wrapped about an electrospun fiber scaffold 120. In some embodiments, the translational and/or rotational movement of the dispenser 106 and the corrugation rod 102 can be controlled to form the monofilament fiber 108 about the electrospun fiber scaffold 120 in a helical shape having a defined number of threads per inch (TPI). It can be desirable to control the TPI of the corrugated electrospun fiber assembly 132 because TPI, in addition to the diameter of the monofilament fiber 108, may dictate the kink radius of the corrugated electrospun fiber assembly 132, as shown below in Table 1. Table 1 illustrates empirical results of testing regarding the relationship between TPI and monofilament thread diameter to kink radius. Accordingly, a desired kink radius can be achieved by selecting a monofilament fiber 108 having the appropriate diameter and controlling the first actuator 104 and/or the second actuator 110 to form the monofilament fiber 108 about the electrospun fiber scaffold 120 in a helical shape having the desired TPI. In some non-limiting examples, the monofilament fiber 108 can be wrapped about the electrospun fiber scaffold 120 in a helical shape having a TPI of about 2, about 4, about 6, about 8, about 10, about 11, about 12, about 13, about 14, about 15, about 16, about 18, about 20, or ranges between any two of these values, including endpoints. In some non-limiting examples, the monofilament fiber 108 can have a thread diameter of about 20 μm, about 40 μm, about 60 μm, about 80 μm, about 100 μm, about 120 μm, about 135 μm, about 140 μm, about 180 μm, about 220 μm, about 260 μm, about 300 μm, about 400 μm, about 500 μm, about 600 μm, about 700 μm, about 800 μm, about 820 μm, about 900 μm, about 1000 μm, or ranges between any two of these values, including endpoints. In one illustrative embodiment, the monofilament fiber 108 can be wrapped around the electrospun fiber scaffold 120 at about 14 TPI with a thread diameter of about 135 μm. In some embodiments, the corrugation system 100 can include a controller 150 that is operably or communicably coupled to the first actuator 104 and/or the second actuator 110. The controller 150 can be configured to control the first actuator 104 and/or the second actuator 110 to correspondingly move the corrugation rod 102 and/or the dispenser 106 to apply the monofilament fiber 108 about the electrospun fiber scaffold 120 at the defined TPI. In some embodiments, the controller 150 can include a control circuit (e.g., an ASIC), a processor (e.g., a CPU) coupled to a memory, a FPGA, and various other hardware, software, and/or firmware components or combinations thereof that are capable of executing logic or instructions.

TABLE 1

| TPI | Thread Diameter (μm) | Kink Radius (mm) |
|---|---|---|
| 6 | 820 | 3 |
| 8 | 135 | 6.65 |
| 8 | 260 | 2.4 |
| 8 | 820 | 4.5 |
| 10 | 260 | 2.38 |
| 10 | 820 | 4.8 |
| 11 | 135 | 4 |
| 15 | 260 | 6.42 |
| 16 | 135 | 3 |
| 18 | 260 | 13.5 |
| 20 | 135 | 4 |

After the monofilament fiber 108 has been wound about the electrospun fiber scaffold 120 to form the wrapped electrospun fiber assembly 130 (FIG. 3A), the wrapped electrospun fiber assembly 130 can be longitudinally compressed from a first length or an uncompressed state, as shown in FIG. 3A, to a second length or a compressed state, as shown in FIG. 3B, to form the corrugated electrospun fiber assembly 132. The action of compressing the wrapped electrospun fiber assembly 130 causes the electrospun fiber scaffold 120 to bulge outwardly between the windings of the monofilament fiber 108 wrapped therearound. This bulging of the electrospun fiber scaffold 120 creates the corrugated surface of the resulting corrugated electrospun fiber assembly 132. In some embodiments, the wrapped electrospun fiber assembly 130 can be either compressed when positioned on the corrugation rod 102 or removed from the corrugation rod and then compressed. In some embodiments, the wrapped electrospun fiber assembly 130 can be compressed manually or via a machine (e.g., a crimping device). In some embodiments, the wrapped electrospun fiber assembly 130 can be compressed to a second or compressed length that is about 35% to about 40% of a first or uncompressed length. In some non-limiting examples, the compressed length can be about 10%, about 20%, about 30%, about 35%, about 40%, about 50%, about 60%, about 70%, about 80%, about 90%, or ranges between any two of these values, including endpoints, of the uncompressed length of the wrapped electrospun fiber assembly 130. In one embodiment, the monofilament fiber 108 can be removed from the corrugated electrospun fiber assembly 132 after its formation.

In some embodiments, the electrospun fiber scaffold 120 may have an inner diameter from about 4 mm to about 25 mm. In some non-limiting examples, the inner diameter of the electrospun fiber scaffold 120 may be about 4 mm, about 6 mm, about 8 mm, about 10 mm, about 15 mm, about 20 mm, about 25 mm, or ranges between any two of these values, including endpoints.

In some embodiments, the helical structure of the corrugation rod 102 may have an outer diameter from about 0.4 mm to about 110 mm. In some non-limiting examples, the outer diameter of the helical structure may be about 0.4 mm, about 0.6 mm, about 0.8 mm, about 1 mm, about 2 mm, about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, or ranges between any two of these values, including endpoints. In one particular embodiment, the corrugation rod 102 may have a diameter from about 5.5 mm to about 5.75 mm.

In some embodiments, the helical structure of the corrugation rod 102 may have a wire gauge from about 40 to about 000 (3/0) (American wire gauge). In some non-limiting examples, the wire gauge of the spiral component may be about 40, about 39, about 38, about 37, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2, about 1, about 0 (1/0), about 00 (2/0), about 000 (3/0), or ranges between any two of these values, including endpoints.

In some embodiments where the corrugation rod 102 includes a helical structure, the helical structure may comprise from about 50 threads per inch to about 4 threads per inch. In some non-limiting examples, the spiral component of the mandrel may comprise about 50 threads per inch, about 45 threads per inch, about 40 threads per inch, about 35 threads per inch, about 30 threads per inch, about 25 threads per inch, about 20 threads per inch, about 15 threads per inch, about 10 threads per inch, about 8 threads per inch, about 7 threads per inch, about 6 threads per inch, about 5 threads per inch, about 4 threads per inch, or ranges between any two of these values, including endpoints.

In some embodiments, the corrugation rod 102 and/or the helical structure thereof may be coated with a non-stick material, such as, for example, aluminum foil, a stainless steel coating, polytetrafluoroethylene, or a combination thereof, before the application of the electrospun fibers. The corrugation rod 102 and/or the helical structure may be fabricated from aluminum, stainless steel, polytetrafluoroethylene, or a combination thereof to provide a non-stick surface on which the electrospun fibers may be deposited. In some embodiments, the corrugation rod 102 and/or the helical structure may be coated with simulated cartilage or other supportive tissue. In some non-limiting examples, the corrugation rod 102 and/or the helical structure may be configured to have a planar surface, a circular surface, an irregular surface, or a substantially cylindrical surface.

In some non-limiting examples, the corrugation rod 102 and/or mandrel may take the form of a bodily tissue or organ, or a portion thereof. In some non-limiting examples, the corrugation rod 102 and/or mandrel may be matched to a subject's specific anatomy. Non-limiting embodiments of such bodily tissues may include a trachea, one or more bronchi, an esophagus, an intestine, a bowel, a ureter, a urethra, a blood vessel, a nerve sheath (including the epineurium or perineurium), a tendon, a ligament, a portion of cartilage, a sphincter, a void, or any other tissue. In one illustrative embodiment, the corrugation rod 102 and/or mandrel can have a about 4 mm to about 7 mm tapered shape configured to form the electrospun structures into a shape suitable to function as a vascular graft.

### Applications of Corrugated Electrospun Fiber Assemblies

Corrugated electrospun fiber assemblies, such as the corrugated electrospun fiber assembly 132 described above, may be particularly useful for biological applications. Without wishing to be bound by theory, a synthetic scaffold that includes electrospun nanofibers may provide an ideal environment for biological cells, perhaps because a typical extracellular matrix configuration is also on the nanometer scale. It may be understood, therefore, that the corrugated electrospun fiber assemblies 132 described herein may be used in a wide variety of biological and surgical applications such as, for example, blood vessels, including peripheral blood vessels, intestines, and other gastrointestinal organs or portions thereof. The corrugated electrospun fiber assemblies 132 may be implanted without any cellular or biological materials, or they may be pre-conditioned to include such materials. In some non-limiting examples, the disclosed corrugated electrospun fiber assemblies 132 may be seeded on both external and luminal surfaces with compatible cells that retain at least some ability to differentiate. In some embodiments, the cells may be autologous cells that may be isolated from the subject (e.g., from the subject's bone marrow) or allogeneic cells that may be isolated from a compatible donor. The seeding process may take place in a bioreactor (e.g., a rotating bioreactor) for a few weeks, days, or hours prior to implantation of the electrospun structure. Additionally, cells may be applied to the corrugated electrospun fiber assemblies 132 immediately before implantation. In some embodiments, one or more growth factors may be added to the composition comprising the electrospun fibers immediately prior to implantation. Corrugated electrospun fiber assemblies 132 incorporating such cells and/or additional chemical factors may then be transplanted or injected into the subject to repair or replace damaged tissue. The subject may be monitored following implantation or injection for signs of rejection or poor function. Any one or more of these procedures may be useful alone or in combination to assist in the preparation and/or transplantation of one or more tissues or a portion of one or more tissues.

It may be appreciated that a variety of biological structures, tissues, and organs may be replaced or repaired by corrugated electrospun fiber assemblies 132. Some non-limiting examples of such structures may include a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, and portions thereof.

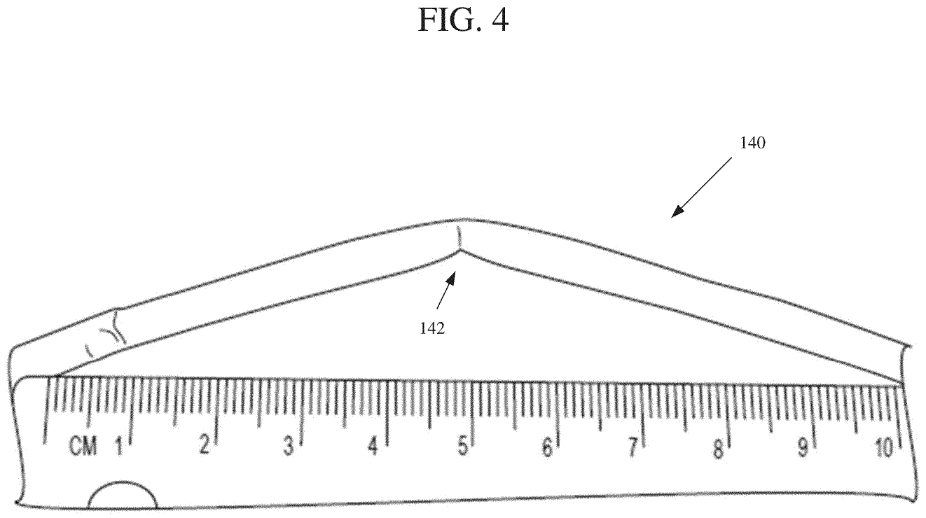

In some embodiments, corrugated electrospun fiber assemblies 132 resulting from the processes described above may comprise an inner wall extending axially and an outer wall adjacent to the inner wall having a plurality of axially adjacent outwardly extending peaks separated by a plurality of valleys. The spacing of these peaks and valleys may be regular or irregular, and the minimum and maximum outer and inner diameters of these peaks and valleys may vary based on the intended application of the corrugated electrospun fiber assembly 132. In some embodiments, the resulting corrugated electrospun fiber assemblies 132 with periodically spaced peaks and valleys may be more flexible than a uniformly shaped electrospun fiber scaffold 140, and may be bent, curved, coiled, or otherwise deformed to a high degree without forming kinks or occlusions, as illustrated in FIGS. 4 and 5. In particular, FIG. 4 illustrates a conventional cylindrical electrospun scaffold 140, which forms a kink 142 after a moderate degree of bending. In comparison, FIG. 5 illustrates an embodiment of a corrugated electrospun fiber assembly 132 described herein, which does not form any kinks even when bent or otherwise manipulated to a much larger degree than the conventional cylindrical electrospun scaffold 140. In some embodiments, the corrugated electrospun fiber assemblies 132 may generally have a spiral or helical configuration. In some embodiments, the spiral configuration of a corrugated electrospun fiber assembly 132 may influence the flow of a substance, such as a fluid, through the electrospun structure. In some embodiments, the spiral configuration of the corrugated electrospun fiber assembly 132 may encourage patency and discourage occlusions, even when the corrugated electrospun fiber assembly is bent, curved, coiled, or otherwise deformed.

In some embodiments, the corrugated electrospun fiber assembly 132 may have one or more wall thicknesses from about 0.01 mm to about 10 mm. In an exemplary embodiment, the corrugated electrospun fiber assembly 132 may have one or more wall thicknesses from about 0.1 mm to about 5 mm. In some non-limiting examples, the one or more wall thicknesses of the corrugated electrospun fiber assembly 132 may be about 0.01 mm, about 1 mm, about 2 mm, about 3 mm, about 4 mm, about 5 mm, about 6 mm, about 7 mm, about 8 mm, about 9 mm, about 10 mm, or any ranges between any two of these values, including endpoints.

Conventional kink-resistant fiber electrospun structures may include rigid spiral components, such as metal springs, rigid plastic helices, and the like, which provide these electrospun structures with their purported kink resistance. It should be appreciated that the spirally configured corrugated electrospun fiber assemblies 132 resulting from the use of the corrugation system 100 disclosed herein may not incorporate rigid spiral components; rather, their carefully controlled fiber compositions and orientations may allow them to have high compliance, favorable mechanical properties, and high kink resistance without the incorporation of such rigid spiral components.

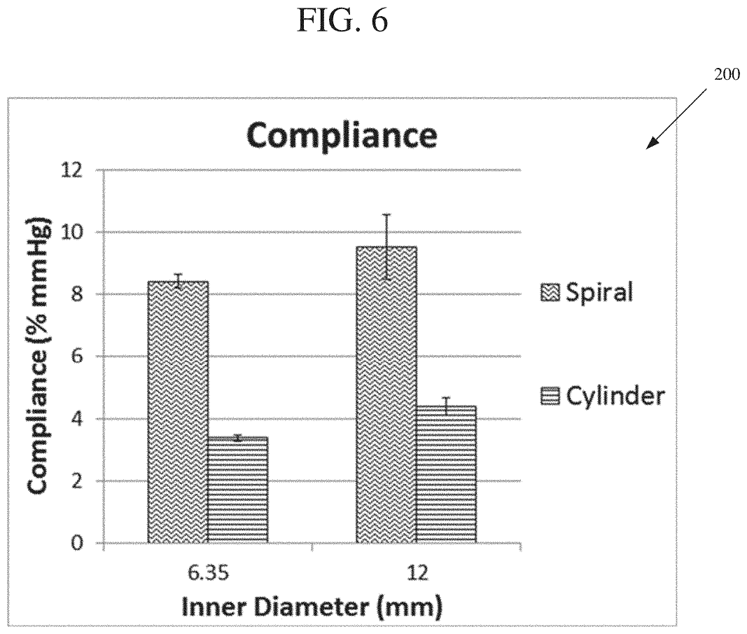

The corrugated electrospun fiber assemblies 132 in accordance with the present disclosure may have significantly increased compliance as compared to that of standard cylindrical electrospun structures with the same diameter and wall thickness, as illustrated in FIG. 6. In particular, FIG. 6 illustrates a bar graph 200 of experimental data indicating that the spirally configured corrugated electrospun fiber assemblies 132 have increased compliance as compared to conventional cylindrical electrospun scaffolds 140 at both an inner diameter of 6.35 mm and 12 mm.

### Example 1

In one example, the compliance of a standard cylindrical electrospun structure was compared to that of a spirally configured electrospun structure in accordance with the present disclosure. For each graft, a 60 cc syringe was filled with water and placed in a syringe pump. The syringe pump was set to a constant flow rate of 5 mL/min. Surgical tubing was connected to the 60 mL syringe, and passed through a pressure transducer. The end of the surgical tubing was connected to a FR18 pediatric Foley catheter. A 2.5 cm long section of the vascular graft was positioned directly over the catheter balloon. The vascular graft section was centered in the field of view of a High Accuracy CCD Micrometer. Pressure and scaffold diameter readings were taken using Labview 2010 software and recorded four times per second. Testing was stopped at the point of failure of the graft, or when the pressure reached 30 psi, due to physical constraints of the catheter, tubing connections, and syringe pump. Compliance (C %) for this test was calculated using the compliance equation below, where PS is the systolic pressure, PD is the diastolic pressure, DS is the diameter at the systolic pressure, and DD is the diameter at the diastolic pressure.

$$C \% = \frac{\frac{D S - D D}{D D}}{P S - P D} * 1 {0}^{4} = \frac{\frac{D S}{DD} - \frac{D D}{DD}}{PS - PD} * 1 {0}^{4} = \frac{\frac{D S}{D D} - 1}{P S - P D} * 1 {0}^{4}$$

FIG. 6 illustrates the results of this testing, and shows that the spirally configured electrospun fiber structures made in accordance with the present disclosure demonstrate significantly increased compliance as compared to that of standard cylindrical electrospun structures with the same diameter and wall thickness.

### Example 2

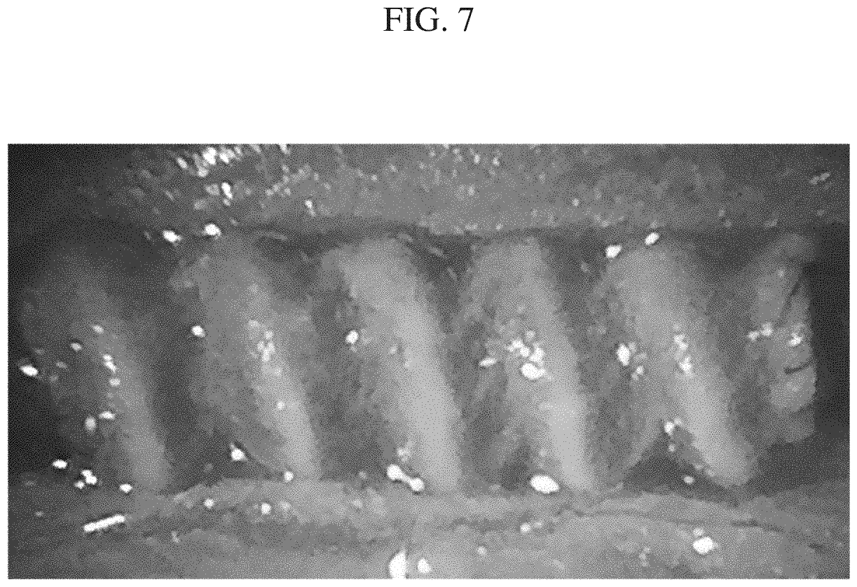

In another example, a spirally configured electrospun structure as described herein was implanted as an interposition infrarenal abdominal aortic (IAA) graft in a murine model. After 4 weeks in vivo, the graft appeared grossly patent, without evidence of aneurysmal dilation or stenosis. FIG. 7 illustrates the graft implanted in vivo in the murine model.

While the present disclosure has been illustrated by the description of exemplary embodiments thereof, and while the embodiments have been described in certain detail, it is not intended to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the general inventive concept.

1. A method of fabricating a corrugated electrospun fiber assembly, the method comprising:
placing an electrospun fiber scaffold on a corrugation rod;
applying a monofilament fiber about the electrospun fiber scaffold and the corrugation rod from a dispenser as the dispenser is translated longitudinally and the corrugation rod is rotated such that the monofilament fiber is wrapped about the electrospun fiber scaffold at a defined threads per inch (TPI) to form a wrapped electrospun fiber assembly; and
longitudinally compressing the corrugated electrospun fiber assembly until it has been compressed from a first length to a second length to form the corrugated electrospun fiber assembly.

2. The method of claim 1, wherein:
the dispenser is coupled to a linear actuator configured to translate the dispenser longitudinally; and
the corrugation rod is coupled to a rotary actuator configured to rotate the corrugation rod.

3. The method of claim 2, wherein applying the monofilament fiber about the electrospun fiber scaffold comprises controlling the linear actuator and the rotary actuator to wrap the monofilament fiber about the electrospun fiber scaffold at the defined TPI and a controlled tension.

4. The method of claim 1, wherein the defined TPI comprises from about 2 TPI to about 20 TPI.

5. The method of claim 1, wherein the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

6. The method of claim 1, further comprising:
electrospinning a polymer to form the electrospun fiber scaffold.

7. The method of claim 6, wherein the polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

8. The method of claim 1, wherein the second length is from about 10% to about 90% of the first length.

9. The method of claim 1, wherein the monofilament fiber has a diameter from about 20 μm to about 1000 μm.

10. A corrugated electrospun fiber assembly comprising:
an electrospun fiber scaffold; and
a monofilament fiber wound about the electrospun fiber scaffold in a helical shape at a defined threads per inch (TPI) and a defined tension such that the electrospun fiber scaffold bulges between windings of the monofilament fiber after being compressed to form a corrugated surface.

11. The corrugated electrospun fiber assembly of claim 10, wherein the defined TPI comprises from about 2 TPI to about 20 TPI.

12. The corrugated electrospun fiber assembly of claim 10, wherein the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

13. The corrugated electrospun fiber assembly of claim 10, wherein the electrospun fiber scaffold comprises a polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

14. The corrugated electrospun fiber assembly of claim 10, wherein the monofilament fiber has a diameter from about 20 μm to about 1000 μm.

15. A system for fabricating a corrugated electrospun fiber assembly, the system comprising:
a corrugation rod comprising a helical structure, the corrugation rod configured to receive an electrospun fiber scaffold thereon;
a rotary actuator configured to rotate the corrugation rod;
a dispenser configured to dispense a monofilament fiber at a controlled tension;
a linear actuator configured to linearly translate the dispenser; and
a controller coupled to the rotary actuator and the linear actuator, the controlled configured to control movement of at least one of the rotary actuator or the linear actuator to cause the monofilament fiber to wind about the electrospun fiber scaffold in a helical shape having a defined threads per inch (TPI).

16. The system of claim 15, wherein the defined TPI comprises from about 2 TPI to about 20 TPI.

17. The system of claim 15, wherein the corrugated electrospun fiber assembly is in a shape of a biological structure selected from the group consisting of a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, or portions thereof.

18. The system of claim 15, wherein the electrospun fiber scaffold comprises a polymer is selected from the group consisting of polypyrrole, polyvinyl pyrrolidone, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, enantiomers thereof, and combinations thereof.

19. The system of claim 15, wherein the monofilament fiber has a diameter from about 20 μm to about 1000 μm.
